(19) United States
(12) Patent Application Publication
(10) Pub. No.: US 2009/0301527 A1
(43) Pub. Date: Dec. 10, 2009

(54) Mechanized Disinfection of Articles

(75) Inventors: Wolfgang Wagemann, Tremsbuttel (DE); Juergen Staffeldt, Winsen (DE); Verona Schmidt, Hamburg (DE)

Correspondence Address: MEDLEN & CARROLL, LLP, 101 HOWARD STREET, SUITE 350, SAN FRANCISCO, CA, 94105, US

(73) Assignee: Chemische Fabrik Dr. Weigert GmbH & Co. KG, Hamburg (DE)

(21) Appl. No.: 11/887,600

(22) PCT Filed: Apr. 6, 2006
(86) PCT No.: PCT/EP2006/003137

§ 371 Date: Jul. 28, 2009

(30) Foreign Application Priority Data
Apr. 6, 2005 (EP) 05007557.1

(51) Int. Cl. B08B 3/00; C11D 3/16

(52) U.S. Cl. 134/26; 510/161; 510/219; 510/218

(57) ABSTRACT

The invention relates to the use of a cleaning agent which comprises at least two different surfactants, selected from the group including cationic, nonionic and amphoteric surfactants, and which has, when diluted in an aqueous solution and ready for use, a pH of at least 10.5. The cleaning agent is used for killing/inactivating microorganisms which are selected from the group including bacteria, viruses and fungi, during mechanized disinfection of articles.

The invention relates to the field of mechanized cleaning and disinfection of articles such as, for example, medical and/or surgical instruments and appliances or kitchenware.

Medical and surgical instruments and appliances must be cleaned and disinfected after use. This can be performed by machine. Disinfection is generally carried out separately from cleaning and comprises either the use of a special agent (chemical or chemothermal disinfection), or thermal disinfection. Also, in the mechanized cleaning of kitchenware (for example in washing drinking glasses), hygiene standards must be met which can make a disinfection step necessary.

The object of the invention is to provide a possibility for disinfecting articles such as medical and/or surgical instruments and appliances in which bacteria, viruses and fungi (yeasts and molds), in particular bacteria and molds, are economically destroyed or inactivated. The invention is intended to be suitable for routine use in mechanized cleaning and disinfection.

The invention therefore relates to the use of a cleaning agent which contains at least two different surfactants selected from the group consisting of cationic, nonionic and amphoteric surfactants, and, diluted ready-to-use in aqueous solution, has a pH of at least 10.5, for destroying/inactivating microorganisms selected from the group consisting of bacteria, viruses and fungi in the mechanized disinfection of articles.

Expressions used in the context of the invention will first be explained.

The expression cleaning agent designates any ready-to-use formulation which is used either directly or diluted with water for cleaning or disinfecting the corresponding instruments. In the context of the invention, the expression cleaning agent includes the expression disinfectant. The cleaning agent can be formulated in solid form, or preferably in liquid form. As cleaning solution, that is to say diluted ready-to-use in aqueous solution, the cleaning agent has a pH of 10.5 or above.

The cleaning agent used according to the invention contains at least two different surfactants from the specified surfactant groups. This means compounds which lower the interfacial tension, that is to say amphiphilic compounds with at least one hydrophobic and one hydrophilic molecule moiety. In the context of the invention, use can be made of all surfactants such as, for example, anionic surfactants (additionally to the surfactant groups mentioned in claim 1), nonionic surfactants, cationic surfactants, amphoteric surfactants and block copolymers (in particular of ethylene oxide and propylene oxide units). By way of example reference is made to Römpp Chemielexikon [Römpp's Chemistry Lexicon], 10th edition, head word “Tenside” [surfactants].

The invention is used in the mechanized cleaning and/or disinfection of articles such as, for example, medical and or surgical instruments and/or appliances. “Mechanized” means that the process preferably proceeds automatically in a dishwashing machine and in the course of cleaning or disinfection no human intervention is required. In particular, according to the invention, a conventional dishwashing and preparation machine for surgical instruments or kitchenware can be employed. It can be used, in particular, for routine daily instrument cleaning or dishwashing.

The expressions “cleaning and/or disinfection” comprise the steps required in the workup of used articles up to a clean state, which, before reuse, can further be followed by a sterilization.

Medical and/or surgical instruments and appliances are all apparatuses and also parts thereof which are used in the medical and hospital sector and are available in principle to mechanized cleaning and disinfection.

The expression kitchenware means all articles, in particular made of glass, porcelain, metal or plastic, which are used in the kitchen sector for preparation and/or consumption of foods. The invention can be used, for example, in the mechanized cleaning and disinfection of drinking glasses. In particular in the mechanized cleaning of drinking glasses in gastronomy, it is required that these are again available for reuse after very short washing times (90 sec to 5 min). The invention here enables sufficient disinfection of the glasses even without the action of heat which is conventional in the prior art. This is particularly advantageous since in this manner, firstly, the washing operation is accelerated, and secondly the glasses are not removed from the dishwashing machine at unsuitably high temperatures for the immediate dispensing of cold drinks.

Destruction/inactivation of microorganisms means that these are sufficiently destroyed in order to enable hazard-free subsequent proper use of surgical instruments and appliances or of kitchenware. Requirements of chemical disinfectants for testing bactericidal activity are laid down, for example in DIN EN 13727 and for testing fungicidal activity, for example, in DIN EN 13624. The virucidal activity is tested, for example, as specified in the directive of the Bundesgesundheitsamt [German Federal Health Office] and the Deutsche Vereinigung zur Bekämpfung von Viruskrankheiten e. V. [German Association for Combating Viral Diseases].

The invention is based on the surprising finding that a combination of alkalinity and at least two different surfactants from the group consisting of cationic, nonionic and amphoteric surfactants has sufficient activity against bacteria, viruses and fungi in the mechanized cleaning and preparation of instruments and also in dishwashing.

When in the context of the invention, at least two different surfactants are spoken of, this means that they are selected from two different groups (cationic, nonionic or amphoteric). At least two of said three groups must therefore be combined in order that the destruction or inactivation according to the invention of microorganisms occurs. Preference is given to a combination of at least three surfactants from all three said groups.

It is possible, in particular according to the invention, to carry out not only the cleaning but also a disinfection using a single agent, so that the complex and expensive storage and also metering of a separate disinfectant can be avoided. In addition, the energy- and time-consuming thermal disinfection at high temperatures, which is stressing, in particular, for sensitive plastic or rubber parts, can also be avoided.

The pH of the cleaning solution diluted ready-to-use is preferably at least 11, further preferably at least 11.5, further preferably at least 12, further preferably at least 12.5. The cleaning agent preferably contains alkali metal hydroxides such as sodium hydroxide, or preferably potassium hydroxide. The use of potassium hydroxide facilitates the provision of a cleaning agent in the form of a concentrate, since potassium hydroxide solutions have a lower tendency to crystallize out at low temperatures than sodium hydroxide solutions.

The preferred alkali metal hydroxide content in the cleaning/disinfection solution diluted ready-to-use is 200 to 10,000 ppm, further preferably 200 to 5000 ppm, further preferably 200 to 2000 ppm. The ppm figure relates to parts by weight.

The cleaning agent can additionally contain alkanolamines.

The content of cationic surfactants in the solution diluted ready-to-use is preferably 15 to 500 ppm, further preferably 15 to 100 ppm, further preferably 15 to 50 ppm. Nonionic surfactants are present in the solution diluted ready-to-use preferably at 15 to 500 ppm, further preferably 15 to 200 ppm, further preferably 25 to 100 ppm. The content of amphoteric surfactants in the solution diluted ready-to-use is preferably 50 to 1000 ppm, further preferably 100 to 500 ppm, further preferably 150 to 300 ppm.

As cationic surfactants, particular preference is given to quaternary ammonium compounds. These, in the context of the combination according to the invention, also act microbicidally at very low use concentrations.

By addition of surfactants to the highly alkaline cleaner solution, the surface tension and interfacial tension can be significantly reduced. In principle, nonionic surfactants such as, for example, fatty alcohols, are most suitable for reducing the surface tension of an aqueous solution. They have the additional advantage that they foam only slightly and thus prevent or reduce the unwanted foam formation in cleaning of medical instruments. Foam formation can, in particular, reduce the circulation pump pressure in the dishwashing machine, impair the cleaning especially of narrow-bore tubes of endoscopes or the like.

The cleaner solution diluted ready-to-use preferably has a surface tension of less than 50 mN/m, preferably less than 40 mN/m, further preferably less than 35 mN/m. The surface tension is determined by what is termed the plate-ring method as specified in DIN 53993.

A further aspect of the invention is avoiding or reducing what is termed redeposition of impurities on the instruments. The expression redeposition denotes the repeated deposition of an impurity already removed from a contaminated surface on another possibly previously uncontaminated surface of the instrument to be cleaned.

The use of surfactants contemplated in the context of the invention already inhibits redeposition, since the surfactants can emulsify separated impurities and thereby keep them in suspension in the aqueous solution. Preference is given in the context of the invention to avoiding or decreasing redeposition, such that the cleaning agent additionally contains hardness dispersants. Hardness dispersants which can be used are, for example, phosphates and polyphosphates, complexing agents or chelating agents or other compounds termed builders. Hardness dispersants support the emulsifying action of surfactants and thereby contribute to preventing redeposition.

An important aspect of the invention is its suitability for routine mechanized cleaning and disinfection. For such a routine cleaning, in the prior art customarily weakly acidic or weakly alkaline (for example enzymatic) cleaners are used, since strongly alkaline solutions can lead to an increased stressing or corrosion and thus wear of various materials and surfaces which are used in medical instruments and appliances. Problems in this respect are, for example, silicone elastomers, chromed instruments, braised joints of silver and tin, glued joints and sealing materials, plastic coatings such as, for example, color codings, fiberglass light conductors and optical surfaces having antireflection coatings. Particular problems are aluminum surfaces, in particular anodized aluminum surfaces, since alkaline solutions show a particular aggressivity toward these. Said problems occur, for example, particularly in the cleaning of endoscopes and components thereof, since here the surfaces to be cleaned have a large variety of materials.

In a preferred embodiment of the invention, therefore the cleaning agent additionally contains corrosion inhibitors. These comprise any substance which, in alkaline solution, inhibits its attack on surfaces, in particular metallic surfaces such as aluminum or anodized aluminum. Suitable inhibitors are, for example, polymeric silicates such as, for example, waterglass, esters of phosphoric acid or the like. Suitable esters of phosphoric acid are mono- and/or diesters of phosphoric acid with aliphatic alcohols of chain length C₁ to C₂₂ and/or aliphatic diols and/or aliphatic polyols of chain length C₂ to C₂₂. According to the invention in this manner, despite the use of highly alkaline cleaner solutions, a gentle action on, for example, anodized aluminum surfaces, is obtained.

According to the invention, from the components of the cleaning agent, preferably a liquid concentrate is formulated which can be diluted with water to give the ready-to-use cleaning solution. In this concentrate, the alkali content (calculated as KOH) is preferably between 2 and 30% by weight, further preferably 15 and 26% by weight. The surfactant content is preferably between 2 and 25% by weight, further preferably 2 and 15% by weight, further preferably 5 and 15% by weight, further preferably 5 and 10% by weight. This concentrate is preferably made up with water to give a ready-to-use solution in concentrations of 0.5 to 5% by volume, preferably 0.5 to 2% by volume, particularly preferably 0.5 to 1.5% by volume.

As mentioned above, the concentrate can contain at least one complexing agent, in particular chelating agent. The complexing agent serves for water softening and can, by complexing alkaline earth ions, improve the cleaning action compared with lime soaps. The complexing agents can be homo-, co- or terpolymers based on acrylic acid or alkali metal salts thereof, in addition phosphonic acids or alkali metal salts thereof, such as, for example, 1-hydroxyethane-1,1-diphosphonic acid, aminotrismethylenephosphonic acid, ethylenediaminotetrakismethylenephosphonic acid, phosphonobutanetricarboxylic acid; tartaric acid, citric acid and gluconic acid; in addition, nitrilotriacetic acid or ethylenediaminetetraacetic acid or salts thereof.

The concentrate can contain nitrilotriacetic acid and/or a salt of this acid, particularly preferably its trisodium salt. NTA addition is advantageous when the concentrate is to be made up with water having a high mineral content (hard water) to give a ready-to-use solution.

Where required, formulation aids (solubilizers) can be added such as, for example, sodium cumenesulfonate, sodium toluenesulfonate, sodium xylenesulfonate, urea, glycols, in particular polypropylene glycols and polyethylene glycols, methylacetamide and fatty alcohols such as, for example, cetyl alcohol.

The enumeration of possible components is not final. In addition, for example wetting agents, emulsifiers, foam-retarding agents or the like can be added. For example, the addition of N-acylglutamate as wetting agent is advantageous.

The time of action of the cleaning agent is according to the invention preferably 1 to 60 min, further preferably 1 to 30 min, further preferably 5 to 30 min, further preferably 10 to 20 min. In particular, in the cleaning/disinfecting of kitchenware, short times of action of, for example, 30 s to 5 min, in particular 60 s to 3 min, can also be provided. Before and/or after the action of the cleaning agent used according to the invention, further precleaning, cleaning, rewashing or rinsing or disinfection steps can be provided. It is preferred first to perform a prewash for removing coarse impurities, then a cleaning/disinfection according to the invention, followed by rewashing with water for removing cleaning agent residues.

Cleaning is preferably carried out according to the invention at a temperature of from room temperature to 93° C., further preferably 40 to 93° C., further preferably 50 to 80° C., particularly preferably 50 to 60° C. A temperature range of from room temperature (18° C.) to 50° C. or room temperature to 40° C. is likewise preferred.

In the mechanized cleaning of medical instruments, particularly preferred temperatures are from 50 to 60° C., in particular about 55° C., and a time of action of from 10 to 20 min, preferably about 10 min. The cleaner concentrate according to example 1 hereinafter is preferably used in the context of mechanized cleaning in a use concentration of about 1 to 2% by volume.

The invention further relates to a process for mechanized cleaning and/or disinfection of articles such as, for example, medical and/or surgical instruments and appliances or kitchenware, in which, in at least one cleaning and/or disinfection step (subsequently and in the patent claims designated a)) destruction/inactivation of said microorganisms is performed by a cleaning agent which contains surfactants and, diluted ready-to-use in aqueous solution, has a pH of at least 10.5. This cleaning or disinfection step a) can be the first step of the process according to the invention. The contaminated instruments are therefore cleaned, without an upstream cleaning step, under what are termed “dirty conditions” and simultaneously disinfected. This procedure has the particular advantage that no contaminated water at all is discharged from the dishwashing machine. If, in contrast, in the first step or the first steps of the process only cleaning without inactivation of the microorganisms is performed, contaminated wastewater escapes from the dishwashing machine and must be if appropriate post-treated, before it can be discharged into the sewage system. In the case of inactivation of the microorganisms under dirty conditions, this first step a) can be followed, for example, by rewashing and subsequent drying.

According to the invention, it is further possible that one or more further prewash and/or cleaning steps are provided upstream of the cleaning or disinfection step a). In this case, cleaning is first performed in the first steps before, under what are termed clean conditions, the disinfection according to the invention takes place. It is particularly preferred when, in the upstream cleaning step, use is made of the same cleaning agent for producing the clean conditions as in the downstream cleaning or disinfection step a). It can be used, in particular in a lower concentration (preferably lower at least by half), so that in the upstream cleaning step essentially cleaning is performed at the lower concentration and not until the downstream step is destruction/inactivation of the microorganisms performed according to the invention. Particularly preferred parameters for the cleaning or disinfection step a) are temperatures of action of about 50 to 60° C., in particular about 55° C., a time of action of 5 to 15 minutes, in particular about 10 minutes, and also an alkali concentration (calculated as KOH) in the solution diluted ready-to-use of 0.05 to about 0.2% by weight, in particular about 0.1% by weight. The total surfactant concentration in the solution diluted ready-to-use can be low and be, for example, about 100 to 200 ppm. Said parameters are established when a cleaning agent concentrate according to example 1 hereinafter is dissolved in water in the ratio 1 to 100.

The use according to the invention and the process enable effective disinfection (destruction/inactivation of said microorganisms) of medical and surgical instruments or kitchenware without the use of separate disinfectants customary in the prior art such as, for example, active chlorine, peroxides, amine active compounds or the like. The invention therefore also relates to the fact that the cleaning agent used according to the invention does not contain such conventional disinfectants. In addition, the invention relates to the fact that a thermal disinfection which is customary in the prior art (for example washing with water heated to 93° C.) can be omitted.

### EXAMPLE 1

A cleaning agent concentrate is prepared according to table 1 hereinafter. The amounts of the starting materials to be used are reported in parts by weight. (Remainder to 100 parts by weight water). Sodium alkylaminodipropionate is an amphoteric surfactant, Bardac LF is a quaternary ammonium compound (QAV) and the fatty alcohol is a nonionic surfactant (n-surfactant).

| | |
|---|---|
| Potassium tripolyphosphate | 21.39 |
| Potassium hydroxide | 10.00 |
| Sodium alkylaminodipropionate | 2.40 |
| Fatty alcohol, C10/12, 4EO, 4-5PO¹ | 0.50 |
| Bardac LF² | 0.25 |
| Sodium waterglass | 27.90 |

¹Block copolymer of C10/C12 fatty alcohols containing 4 ethylene oxide and 4-5 propylene oxide units.
²Cationic surfactant (dioctyldimethylammonium chloride)

### EXAMPLES 2 to 19

The composition of the concentrate of example 1 was varied according to the details in table 2 hereinafter. Table 2 mentions only the changes with respect to the formula of example 1, all components not mentioned are present in examples 2 to 19 in the same quantitative proportion as in example 1. In each case, the mixture is made up to 100 parts by weight with water. In examples 14 to 16, the concentrate additionally contains 2% by weight isopropanol as solubilizer for the modified and more sparingly soluble amphoteric surfactants.

TABLE 2

| | |
|---|---|
| Example 2 | without Bardac LF (QAV) |
| Example 3 | without fatty alcohol (n-surfactant) |
| Example 4 | without sodium alkylaminodipropionate (ampho- |
| | surfactant) |
| Example 5 | potassium hydroxide content reduced to 5% by |
| | weight |
| Example 6 | Bardac LF replaced by Bardac 2270E |
| | (didecyldimethylammonium chloride) |
| Example 7 | Bardac LF content reduced to 0.25% by weight |
| Example 8 | Bardac LF content increased to 0.375% by weight |
| Example 9 | fatty alcohol content reduced to 0.25% by |
| | weight |
| Example 10 | fatty alcohol content increased to 0.75% by |
| | weight |
| Example 11 | fatty alcohol, C10/12, 4EO, 4-5PO replaced by |
| | oleyl-cetyl alcohol containing 5EO (Eumulgin EP |
| | 5 L-V) |
| Example 12 | fatty alcohol, C10/12, 4EO, 4-5PO replaced by |
| | end group-capped C12/18 5EO (Dehypon LT 054) |
| Example 13 | fatty alcohol, C10/12, 4EO, 4-5PO replaced by |
| | N-octylpyrrolidone |
| Example 14 | Bardac LF replaced by N,N-didecyl- |
| | N-methylpoly(oxyethyl)ammonium propionate |
| | (Bardap 26) |
| Example 15 | Bardac LF replaced by lauryldimethylbenzyl- |
| | ammonium chloride/lauryldimethylethylbenzyl- |
| | ammonium chloride (BTC 2125 M) |
| Example 16 | Bardac LF replaced by N-alkyl(C12/14)-N-benzyl- |
| | N,N-dimethylammonium chloride (Barquat LB 50) |
| Example 17 | sodium alkylaminodipropionate replaced by |
| | alkylaminoacetic acid mixture (Ampholyt 51/27) |
| Example 18 | sodium alkylaminodipropionate replaced by |
| | Na-Capr•amphppropionate (Rewoteric AM-VSF) |
| Example 19 | sodium alkylaminodipropionate replaced by fatty |
| | acid amidopropyl betaine (Tegotens B 810) |

### COMPARATIVE EXAMPLES 1 to 6

Cleaning concentrates not according to the invention are prepared according to table 3 hereinafter. Table 3 mentions only the changes with respect to the formula of example 1, all components not mentioned are present in comparative examples 1 to 6 in the same quantitative proportion as in example 1. In each case, the mixture is made up to 100 parts by weight with water.

TABLE 3

| | |
|---|---|
| Comparative example 1 | without surfactants (all 3 |
| | surfactants omitted) |
| Comparative example 2 | without Bardac LF and fatty |
| | alcohol |
| Comparative example 3 | without fatty alcohol and sodium |
| | alkylaminodipropionate |
| Comparative example 4 | without Bardac LF and sodium |
| | alkylaminodipropionate |
| Comparative example 5 | potassium hydroxide content |
| | reduced to 1% by weight |
| Comparative example 6 | without potassium hydroxide |

### EXAMPLE 20

The cleaning agent concentrates according to examples 1 to 19 and comparative examples 1 to 6 are tested for bactericidal activity. The standards mentioned hereinafter, and in particular the conditions and processes described there for testing activity are hereby explicitly also incorporated in the present application by reference. In testing the destruction/inactivation of bacteria, the reduction factor of the microorganisms mentioned in table 4 was measured of an aqueous solution of the concentrates at the time of action and temperature reported. The concentration of the cleaning agent used is reported in % by volume. These concentrations, time of action and temperatures correspond approximately to the typical conditions in mechanized cleaning. The reduction factors achieved are reported in steps of logarithms to base ten (log steps).

TABLE 4

| | | | | | Mykobakterium terrae | | | | Mykobakterium terrae | | | | | |
|---|---|---|---|---|---|---|---|---|---|---|---|---|---|---|
| | | | | | Test according | | | | Test according | | | | Enterococcus hirae | |
| | | | | | to standard | | | | to standard | | | | Test as specified in | |
| | | | | | methods of DGHM, test | | | | methods of DGHM, test | | | | DIN EN 13727, test | |
| | Active compound | | | | temperature 55° C., time | | | | temperature 55° C., time | | | | temperature 20° C., | |
| | concentration | | | | of action 10 min | | | | of action 10 min | | | | time of action 5 min | |
| | in 1% strength | | | | low pollution | | | | high pollution | | | | low pollution | |
| | solution [ppm] | | | | 1.0% | 1.5% | 2.0% | 2.5% | 1.0% | 1.5% | 2.0% | 2.5% | 1.0% | 2.0% |
| | | | ampho- | | by | by | by | by | by | by | by | by | by | by |
| | QAV | n-surfactant | surfactant | KOH | vol. | vol. | vol. | vol. | vol. | vol. | vol. | vol. | vol. | vol. |
| Ex. 1 | 25 | 50 | 240 | 1000 | 3.22 | 4.10 | 6.10 | | 3.41 | 4.66 | 4.54 | 5.14 | | |
| Ex. 2 | — | 50 | 240 | 1000 | — | 3.38 | 3.40 | — | — | 3.42 | 3.78 | 3.79 | — | — |
| Ex. 3 | 25 | — | 240 | 1000 | — | 4.15 | 3.80 | — | — | 3.19 | 3.36 | 3.84 | — | — |
| Ex. 4 | 25 | 50 | — | 1000 | — | 3.61 | 3.92 | — | — | 3.38 | 3.54 | 3.66 | — | — |
| Ex. 5 | 25 | 50 | 240 | 500 | — | 3.80 | 4.32 | — | — | 3.80 | 4.19 | 4.36 | — | — |
| Ex. 6 | 25 | 50 | 240 | 1000 | — | 4.80 | 4.80 | — | — | 4.36 | 4.54 | 4.66 | — | — |
| Ex. 7 | 12.5 | 50 | 240 | 1000 | 1.52 | 3.58 | 4.06 | — | 2.33 | 3.28 | 3.38 | — | — | — |
| Ex. 8 | 37.5 | 50 | 240 | 1000 | 3.30 | 3.76 | 3.95 | — | 3.55 | 3.71 | 4.03 | — | — | — |
| Ex. 9 | 25 | 25 | 240 | 1000 | 3.07 | 3.80 | 4.06 | — | 3.27 | 3.57 | 3.88 | — | — | — |
| Ex. 10 | 25 | 75 | 240 | 1000 | 3.47 | 3.70 | 3.99 | — | 3.43 | 3.65 | 4.14 | — | — | — |
| Comp. | — | — | — | 1000 | — | 1.67 | 1.87 | — | — | 1.94 | 2.04 | 2.10 | — | — |
| ex. 1 | | | | | | | | | | | | | | |
| Comp. | — | — | 240 | 1000 | <1.22 | <1.22 | <1.22 | — | <1.05 | <1.05 | <1.05 | — | — | — |
| ex. 2 | | | | | | | | | | | | | | |
| Comp. | 25 | — | — | 1000 | <1.22 | 1.77 | 1.93 | — | <1.05 | 1.59 | 1.60 | — | — | — |
| ex. 3 | | | | | | | | | | | | | | |
| Comp. | — | 50 | — | 1000 | 1.74 | 1.86 | 1.96 | — | <1.05 | 1.61 | 1.80 | — | — | — |
| ex. 4 | | | | | | | | | | | | | | |
| Comp. | 25 | 50 | 240 | 100 | — | — | — | — | — | — | — | — | 1.17 | 4.74 |
| ex. 5 | | | | | | | | | | | | | | |
| Comp. | 25 | 50 | 240 | — | — | — | — | — | — | — | — | — | 0.55 | 2.76 |
| ex. 6 | | | | | | | | | | | | | | |
| Ex. 11 | 25 | 50 | 240 | 1000 | — | — | — | — | — | — | — | — | 2.62 | >5.23 |
| Ex. 12 | 25 | 50 | 240 | 1000 | — | — | — | — | — | — | — | — | 3.23 | >5.23 |
| Ex. 13 | 25 | 50 | 240 | 1000 | — | — | — | — | — | — | — | — | 3.83 | >5.23 |
| Ex. 14 | 25 | 50 | 240 | 1000 | — | — | — | — | — | — | — | — | 4.39 | >5.23 |
| Ex. 15 | 25 | 50 | 240 | 1000 | — | — | — | — | — | — | — | — | >5.23 | >5.23 |
| Ex. 16 | 25 | 50 | 240 | 1000 | — | — | — | — | — | — | — | — | >5.23 | >5.23 |
| Ex. 17 | 25 | 50 | 240 | 1000 | — | — | — | — | — | — | — | — | >5.23 | >5.23 |
| Ex. 18 | 25 | 50 | 240 | 1000 | — | — | — | — | — | — | — | — | 4.99 | >5.23 |
| Ex. 19 | 25 | 50 | 240 | 1000 | — | — | — | — | — | — | — | — | 4.19 | 4.51 |

In table 4, “low pollution” means that the protein loading of the test suspensions used is low (0.03% bovine serum albumin). This corresponds to inactivation of the microorganisms under what are termed clean conditions, under which, before disinfection, a separate cleaning step has been performed. Correspondingly, “high pollution” means a high loading with protein residues (0.3% bovine serum albumin and 0.3% sheep erythrocytes); this corresponds to disinfection (and simultaneous cleaning) under what are termed dirty conditions, under which no separated upstream cleaning or prewash is performed.

The experiments of table 4 show that reduction of the microorganisms by at least the factor 10³ (3 log steps) may be achieved over a broad variation of amount and type of components. The comparative examples in turn verify that such an inactivation does not occur if less than two surfactants from said three surfactant groups are present or the alkalinity is insufficient. Still better inactivation of the microorganisms results when all three surfactant groups are combined.

In the context of the invention, reduction of the microorganisms by at least 3 log steps is preferred. A reduction by at least 4 log steps is further preferred. This reduction by 4 log steps can be preferred, in particular in the case of fungi. A reduction by 5 log steps is further preferred, in particular in the case of bacteria.

### EXAMPLE 21

In this example, the activity of the formula of example 1 is demonstrated against various microorganisms and for various times of action and concentrations. In table 5 hereinafter, the details of dirt loading of the test bodies and of the cleaner concentrations used correspond to the abbreviations used in table 4. From the table it may be seen that example 1 effects an inactivation by at least the factor 10⁴ of the test fungi Candida albicans and Aspergillus niger.

TABLE 5
Mycobacterium terrae
Test as specified in DIN EN 14348, test
temperature 55° C., time of action 10 min

| | | |
|---|---|---|
| low pollution | 1.0% by volume | 5.60 |
| | 1.5% by volume | 6.18 |
| | 2.0% by volume | 6.78 |
| high pollution | 1.5% by volume | 5.32 |
| | 2.0% by volume | 5.08 |

E. hirae
Test as specified in DIN EN 13727, test
temperature 55° C., time of action 5 min

| | | |
|---|---|---|
| low pollution | 1.0% by volume | >5.23 |
| high pollution | 1.0% by volume | >5.23 |

Ps. aerug.
Test as specified in DIN EN 13727, test
temperature 55° C., time of action 5 min

| | | |
|---|---|---|
| low pollution | 1.0% by volume | >5.04 |
| high pollution | 1.0% by volume | >5.04 |

Staph. aur.
Test as specified in DIN EN 13727, test
temperature 55° C., time of action 5 min

| | | |
|---|---|---|
| low pollution | 1.0% by volume | >5.28 |
| high pollution | 1.0% by volume | >5.28 |

C. albic.
Test as specified in DIN EN 13624, test temperature
55° C., time of action 5 min at test concentration 1.0%
by volume; 10 min at test concentration 2.0% by volume

| | | |
|---|---|---|
| low pollution | 1.0% by volume | >4.15 |
| | 2.0% by volume | >4.26 |
| high pollution | 2.0% by volume | >4.15 |

Asp. niger
Test as specified in DIN EN 13624, test temperature
55° C., time of action 5 min at test concentration 1.0%
by volume; 10 min at test concentration 2.0% by volume

| | | |
|---|---|---|
| low pollution | 1.0% by volume | 3.83 |
| | 2.0% by volume | >4.20 |
| high pollution | 2.0% by volume | >4.28 |

### EXAMPLE 22

In this example, two program sequences are specified for cleaning and disinfecting medical and surgical instruments in a customary one-tank washing machine.

Program Sequence 1:
precleaning with cold water, 3 min,
cleaning with a 0.3% strength by volume aqueous solution of the formula according to example 1 at 55° C., holding time 3 min,
cleaning/disinfection with a 1% strength by volume aqueous solution of the formula according to example 1 at 55° C., holding time 10 min,
rewashing with water with addition of an acidic neutralizing agent based on citric acid,
intermediate rinse with cold water,
final rinse with water, heating to 55° C. with a time of action of 1 min,
drying with hot air.

In this process sequence, the disinfection is carried out under what are termed clean conditions, that is to say before the actual disinfection, first cleaning is carried out using a lower concentration of the cleaning agent according to example 1. The cold water prewash provided in the program sequence can optionally be omitted entirely. The rewashing after the disinfection step can also optionally proceed without neutralization agent if sufficient water for rinsing off the alkaline formula according to example 1 is used.

Program Sequence 2:
precleaning with cold water, 3 min,
cleaning/disinfection with a 1% strength by volume aqueous solution of the formula according to example 1 at 55° C., holding time 10 min,
rewashing with water with addition of an acidic neutralizing agent based on citric acid,
intermediate rinse with cold water,
final rinse with water, heating to 55° C. with time of action of 1 min,
drying with hot air.

In this program sequence, the disinfection proceeds under dirty conditions, that is to say cleaning and disinfection proceed simultaneously in a single step. Optionally, the cold water precleaning can be omitted. This has, in the cleaning under dirty conditions, the particular advantage that then all of the washing water exiting from the washing machine is not contaminated, since the first cleaner solution coming into contact with the contaminated instruments already disinfects, and thus eliminates contaminations by microorganisms.

According to the invention, a final thermal disinfection is not required in any of the program sequences. This shortens the program sequences, since heating to the thermal disinfection temperature of, for example, 93° C., costs times, in addition energy is saved and also the wear of sensitive instruments (in particular plastic and rubber parts) due to thermal stress is decreased.

### EXAMPLE 23

For testing the material care of anodized aluminum surfaces, anodized aluminum plates are exposed in the Miele dishwashing machine G7736 for 10 min at 55° C. to a cleaning medium. Both new colorless and blue anodized aluminum plates are used. The cleaning medium is 0.1M NaOH having a pH of 12.7, or a 1% strength by volume cleaner solution of the cleaning agent concentrate according to example 1. Subsequently, the plates are inspected visually. In the case of the NaOH-treated plates, the anodized layer is significantly eroded. In contrast thereto, the plates treated with the cleaner have no visible damage to the anodized layer.

1. The use of a cleaning agent which contains at least two different surfactants selected from the group consisting of cationic, nonionic and amphoteric surfactants, and, diluted ready-to-use in aqueous solution, has a pH of at least 10.5, for destroying/inactivating microorganisms selected from the group consisting of bacteria, viruses and fungi in the mechanized disinfection of articles.

2. The use as claimed in claim 1, characterized in that the articles are medical and/or surgical instruments or appliances.

3. The use as claimed in claim 1, characterized in that the articles are kitchenware.

4. The use as claimed in claim 3, characterized in that the kitchenware is glass kitchenware.

5. The use as claimed in one of claims 1 to 4, characterized in that the pH is at least 11, preferably at least 11.5, preferably at least 12, further preferably at least 12.5.

6. The use as claimed in one of claims 1 to 5, characterized in that the cleaning agent contains alkali metal hydroxides.

7. The use as claimed in claim 6, characterized in that, as alkali metal hydroxide, use is made of KOH.

8. The use as claimed in claim 6 or 7, characterized in that, in the solution diluted ready-to-use, the alkali metal hydroxide content is 200-10,000 ppm, preferably 200 to 5000 ppm, further preferably 200 to 2000 ppm.

9. The use as claimed in one of claims 1 to 8, characterized in that the cleaning agent contains alkanolamines.

10. The use as claimed in one of claims 1 to 9, characterized in that the cleaning agent contains cationic, nonionic and amphoteric surfactants.

11. The use as claimed in one of claims 1 to 10, characterized in that, in the solution diluted ready-to-use, the content of cationic surfactants is 15-500 ppm, preferably 15-100 ppm, further preferably 15-50 ppm.

12. The use as claimed in one of claims 1 to 11, characterized in that, in the solution diluted ready-to-use, the content of nonionic surfactants is 15-500 ppm, preferably 15-200 ppm, further preferably 25-100 ppm.

13. The use as claimed in one of claims 1 to 12, characterized in that, in the solution diluted ready-to-use, the content of amphoteric surfactants is 50-1000 ppm, preferably 100-500 ppm, further preferably 150-300 ppm.

14. The use as claimed in one of claims 1 to 12, characterized in that the cationic surfactants are quaternary ammonium compounds.

15. The use as claimed in one of claims 1 to 14, characterized in that the cleaning agent, diluted ready-to-use, has a surface tension of less than 50 mN/m, preferably less than 40 mN/m, further preferably less than 35 mN/m.

16. The use as claimed in one of claims 1 to 15, characterized in that the cleaning agent contains hardness dispersants agents.

17. The use as claimed in claim 16, characterized in that the cleaning agent contains phosphates and/or polyphosphates.

18. The use as claimed in one of claims 1 to 17, characterized in that the cleaning agent contains corrosion inhibitors.

19. The use as claimed in claim 18, characterized in that the corrosion inhibitors are selected from the group consisting of polymeric silicates and esters of phosphoric acid.

20. The use as claimed in one of claims 1 to 19, characterized in that the time of action of the cleaning agent is 1 to 60 min, preferably 1 to 30 min, further preferably 5 to 30 min, further preferably 10 to 20 min.

21. The use as claimed in one of claims 1 to 20, characterized in that the cleaning takes place at a temperature from room temperature to 93° C., preferably 40 to 93° C., further preferably 50 to 80° C., further preferably 50 to 60° C.

22. A process for mechanized cleaning and/or disinfection of articles, characterized in that, in at least one cleaning and/or disinfection step a), destruction/inactivation of microorganisms selected from the group consisting of bacteria, viruses and fungi is performed by a cleaning agent which contains at least two different surfactants selected from the group consisting of cationic, nonionic and amphoteric surfactants and, diluted ready-to-use in aqueous dilution, has a pH of at least 10.5.

23. The process as claimed in claim 22, characterized in that the cleaning and/or disinfection step a) is the first step of the process.

24. The process as claimed in claim 22, characterized in that a cleaning step is provided upstream of the cleaning and/or disinfection step a).

25. The process as claimed in claim 24, characterized in that, in the upstream cleaning step, the same cleaning agent is used as in the cleaning and/or disinfection step a).

26. The process as claimed in claim 25, characterized in that, in the upstream cleaning step, the concentration of the cleaning agent is lower than in the cleaning and/or disinfection step a).

27. The process as claimed in claim 26, characterized in that, in the upstream cleaning step, the concentration of the cleaning agent is lower by at least half than in the cleaning and/or disinfection step a).

28. The process as claimed in one of claims 22 to 27, characterized in that it comprises no thermal disinfection step.
